(19) United States
(12) Patent Application Publication
(10) Pub. No.: US 2009/0092647 A1
(43) Pub. Date: Apr. 9, 2009

(54) POLYURETHANE FOAMS FOR WOUND MANAGEMENT

(75) Inventors: Jan Schoenberger, Solingen (DE); Michael Mager, Leverkusen (DE); Sebastian Doerr, Duesseldorf (DE); Thorsten Rische, Unna (DE); Michael Heckes, Krefeld (DE); Daniel Rudhardt, Koeln (DE); Rolf Gertzmann, Leverkusen (DE); Melita Dietze, Erkrath (DE); Burkhard Fugmann, Ratingen (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/245,286

(22) Filed: Oct. 3, 2008

(30) Foreign Application Priority Data
Oct. 5, 2007 (EP) 07019563.1

(51) Int. Cl. A61K 31/155; C08G 18/02; A01N 25/10

(52) U.S. Cl. 424/409; 521/170; 521/163; 521/137; 424/486; 514/635

(57) ABSTRACT

The invention relates to a process for producing polyurethane foams for wound management wherein a composition containing a polyurethane dispersion and inorganic, cationic coagulants is frothed and dried.

### RELATED APPLICATIONS

This application claims benefit to European Patent Application No. 07019563.1, filed Oct. 5, 2007, which is incorporated herein by reference in its entirety for all useful purposes.

### BACKGROUND OF THE INVENTION

The invention relates to a process for producing polyurethane foams for wound management wherein a composition containing a polyurethane dispersion and inorganic, cationic coagulants is frothed and dried.

The use of wound dressings made of foams for treating weeping wounds is prior art. Owing to their high absorbency and their good mechanical properties, polyurethane foams produced by reaction of mixtures of diisocyanates and polyols or NCO-functional polyurethane prepolymers with water in the presence of certain catalysts and also (foam) additives are generally used. Aromatic diisocyanates are generally employed, since they are best foamable. Numerous forms of these processes are known, for example described in U.S. Pat. No. 3,978,266, U.S. Pat. No. 3,975,567 and EP-A 0 059 048. However, the aforementioned processes have the disadvantage that they require the use of reactive mixtures, containing diisocyanates or corresponding NCO-functional prepolymers, whose handling is technically inconvenient and costly, since appropriate protective measures are necessary for example.

One alternative to the above-described process, in which diisocyanates or NCO-functional polyurethane prepolymers are utilized, is a process based on polyurethane dispersions (which are essentially free of isocyanate groups) into which air is incorporated by vigorous stirring in the presence of suitable (foam) additives. So-called mechanical polyurethane foams are obtained after drying and curing. In connection with wound dressings, such foams are described in EP-A 0 235 949 and EP-A 0 246 723, the foam either having a self-adherent polymer added to it, or being applied to a film of a self-adherent polymer. In addition, the examples recited in EP-A 0 235 949 and EP-A 0 246 723 mandate the use as crosslinkers of polyaziridines which are no longer acceptable because of their toxicity. Moreover, crosslinking requires the use of high baking temperatures, reported to be in the range from 100° C. to 170° C. U.S. Pat. No. 4,655,210 describes the use of the aforementioned mechanical foams for wound dressings having a specific construction made up of backing, foam and skin contact layer. The foams produced according to the processes described in EP-A 0 235 949 and EP-A 0 246 723, moreover, have the immense disadvantage that the foams obtained are only minimally open-cell, reducing the absorbence of physiological saline and also the water or moisture vapour transmission rate.

The management of wounds having a complex topology or the coverage of particularly deep wounds is very difficult using ready-to-use, industrially manufactured sheetlike wound dressings, since optimal covering of the wound surface is generally not accomplished, retarding the healing process. To achieve better covering of deep wounds, it has been proposed to use granules of microporous polyurethanes instead of compact wound dressings (EP-A-0 171 268). However, not even this achieves optimal covering of the wound.

The application of a (flowable) composition which optimally conforms to the wound shape would eliminate the disadvantages of sheetlike wound dressings. The two processes described above, which utilize either diisocyanates/NCO-functional polyurethane prepolymers or polyurethane dispersions in combination with polyaziridines to produce polyurethane foams, cannot be used for this, however: reactive compositions containing free isocyanate groups cannot be applied directly to the skin, even though this has been variously proposed (see WO 02/26848 for example). But even use of polyurethane dispersions with polyaziridines as crosslinkers is out of the question today because the crosslinker has properties which are not generally recognized as safe by toxicologists.

A process for the rapid consolidation of mechanically foamed polyurethane dispersion is so far not known, even though the production of polyurethane films, i.e. non-porous materials, by, for example, coagulation with inorganic salts is a technically common process.

The present invention therefore has for its object to provide polyurethane foams for wound management by using a composition which is free of isocyanate groups. The production of the polyurethane foam shall in principle also be able to be carried out under ambient conditions, in which case the polyurethane foams, as well as good mechanical properties, formed shall have a high absorbence of physiological saline and a high water and moisture vapour transmission rate. This requires that the polyurethane foam have a certain open-cell content. Moreover, the composition shall be suitable for direct application to the skin, for example by spraying or pouring, in order that the wound may be optimally covered with the polyurethane foam; rapid drying is essential for this.

### EMBODIMENTS OF THE INVENTION

An embodiment of the present invention is a process for producing foamed articles made of polyurethane foams comprising frothing and drying a composition comprising an aqueous, anionically hydrophilicized, polyurethane dispersion (I) and an inorganic cationic coagulant (II).

Another embodiment of the present invention is the above process, wherein said foamed article is a wound dressing.

Another embodiment of the present invention is the above process, wherein said aqueous, anionically hydrophilicized, polyurethane dispersion (I) is prepared by A) producing isocyanate-functional prepolymers from A1) organic polyisocyanates; A2) polymeric polyols having number-average molecular weights in the range from 400 to 8000 g/mol and OH functionalities in the range from 1.5 to 6; and A3) optionally hydroxyl-functional compounds having molecular weights in the range from 62 to 399 g/mol; and A4) optionally isocyanate-reactive, anionic or potentially anionic and optionally nonionic hydrophilicizing agents and B) wholly or partly reacting the free NCO groups of said isocyanate-functional prepolymer B1) optionally with amino-functional compounds having molecular weights in the range from 32 to 400 g/mol; and B2) with amino-functional, anionic or potentially anionic hydrophilicizing agents; by chain extension; wherein said isocyanate-functional prepolymers are dispersed in water before, during, or after step B), and wherein any potentially ionic groups present are converted into the ionic form by partial or complete reaction with a neutralizing agent.

Another embodiment of the present invention is the above process, wherein A1) is 1,6-hexamethylene diisocyanate, isophorone diisocyanate, the isomeric bis-(4,4′-isocyanatocyclohexyl)methanes, or mixtures thereof, and A2) is a mixture of polycarbonate polyols and polytetramethylene glycol polyols, wherein the proportion of A2) which is contributed by the sum total of the polycarbonate and polytetramethylene glycol polyether polyols is at least 70% by weight.

Another embodiment of the present invention is the above process, wherein said inorganic cationic coagulant (II) is a water-soluble inorganic salt.

Another embodiment of the present invention is the above process, wherein said water-soluble inorganic salt is an alkaline earth metal salt.

Another embodiment of the present invention is the above process, wherein said water-soluble inorganic salt is magnesium chloride, calcium chloride, or a mixture thereof.

Another embodiment of the present invention is the above process, further comprising auxiliary and additive materials (III).

Another embodiment of the present invention is the above process, wherein said auxiliary and additive materials (III) foam formers and stabilizers selected from the group consisting of fatty acid amides, sulphosuccinamides, hydrocarbyl sulphonates, hydrocarbyl sulphates, fatty acid salts, EO-PO block copolymers and/or alkyl polyglycoside.

Another embodiment of the present invention is the above process, wherein said foam formers and stabilizers are EO-PO block copolymers.

Another embodiment of the present invention is the above process, wherein said, further comprising active components selected from the group consisting of antiseptics, growth factors, protease inhibitors, and nonsteroidal anti-inflammatories/opiates.

Another embodiment of the present invention is the above process, wherein said active component is an antiseptic biguanide and/or its salt.

Yet another embodiment of the present invention is a foamed article produced by the process of claim 1.

Another embodiment of the present invention is the above foamed article, wherein said foamed article has a microporous, open-cell structure and a density of below 0.4 g/cm³ in the dried state.

Another embodiment of the present invention is the above foamed article, wherein said foamed article has a DIN EN 13726-1 Part 3.2 physiological saline absorbency of 100 to 1500% (mass of liquid taken up, based on the mass of dry foam) and a DIN EN 13726-2 Part 3.2 water vapour transmission rate in the range from 500 to 8000 g/24 h*m².

Another embodiment of the present invention is the above foamed article, wherein said foamed article is a wound dressing.

Yet another embodiment of the present invention is a composition comprising an aqueous, anionically hydrophilicized polyurethane dispersion (I) and an inorganic cationic coagulant (II).

Another embodiment of the present invention is the above composition, further comprising an active component selected from the group consisting of antiseptics, growth factors, protease inhibitors, and nonsteroidal anti-inflammatories/opiates.

Another embodiment of the present invention is the above composition, wherein said active component is an antiseptic biguanide and/or its salt.

### DESCRIPTION OF THE INVENTION

It has now been found that compositions containing polyurethane dispersions which are free of isocyanate groups and inorganic, cationic coagulants, can be used to produce at ambient conditions polyurethane foams having good mechanical properties, a high absorbence of physiological saline and a high water and moisture vapour transmission rate. The polyurethane foams exhibit, at least to some extent, an open-cell pore structure. The flowable compositions, moreover, can be applied directly to the skin by spraying or pouring.

The present invention accordingly provides a process for producing foamed articles, wound dressings preferably made of polyurethane foams which comprises a composition containing aqueous, anionically hydrophilicized polyurethane dispersions (I) and anionic cationic coagulants (II) being frothed and dried.

Polyurethane foam wound dressings for the purposes of the present invention are porous materials, preferably having at least some open-cell content, which consist essentially of polyurethanes and protect wounds against germs and environmental influences like a sterile covering, have a fast and high absorbence of physiological saline or to be more precise wound fluid, have a suitable permeability for moisture to ensure a suitable wound climate, and have sufficient mechanical strength.

The aqueous, anionically hydrophilicized polyurethane dispersions (I) included in the compositions which are essential to the present invention are obtainable by

A) isocyanate-functional prepolymers being produced from
A1) organic polyisocyanates
A2) polymeric polyols having number average molecular weights in the range from 400 to 8000 g/mol, preferably in the range from 400 to 6000 g/mol and more preferably in the range from 600 to 3000 g/mol, and OH functionalities in the range from 1.5 to 6, preferably in the range from 1.8 to 3, more preferably in the range from 1.9 to 2.1, and
A3) optionally hydroxyl-functional compounds having molecular weights in the range from 62 to 399 g/mol and
A4) optionally isocyanate-reactive, anionic or potentially anionic and/or optionally nonionic hydrophilicizing agents and
   B) its free NCO groups then being wholly or partly reacted
B1) optionally with amino-functional compounds having molecular weights in the range from 32 to 400 g/mol and
B2) with isocyanate-reactive, preferably amino-functional, anionic or potentially anionic hydrophilicizing agents
   by chain extension, and the prepolymers being dispersed in water before, during or after step B), any potentially ionic groups present being converted into the ionic form by partial or complete reaction with a neutralizing agent.

To achieve anionic hydrophilicization, A4) and/or B2) shall utilize hydrophilicizing agents that have at least one NCO-reactive group such as amino, hydroxyl or thiol groups and additionally have —COO⁻ or —SO₃⁻ or —PO₃²⁻ as anionic groups or their wholly or partly protonated acid forms as potentially anionic groups.

Preferred aqueous, anionic polyurethane dispersions (I) have a low degree of hydrophilic anionic groups, preferably from 0.1 to 15 milliequivalents per 100 g of solid resin.

To achieve good sedimentation stability, the number average particle size of the specific polyurethane dispersions is preferably less than 750 nm and more preferably less than 500 nm, determined by laser correlation spectroscopy.

The ratio of NCO groups of compounds of component A1) to NCO-reactive groups such as amino, hydroxyl or thiol groups of compounds of components A2) to A4) is in the range from 1.05 to 3.5, preferably in the range from 1.2 to 3.0 and more preferably in the range from 1.3 to 2.5 to prepare the NCO-functional prepolymer.

The amino-functional compounds in stage B) are used in such an amount that the equivalent ratio of isocyanate-reactive amino groups of these compounds to the free isocyanate groups of the prepolymer is in the range from 40 to 150%, preferably between 50 and 125% and more preferably between 60 and 120%.

Suitable polyisocyanates for component A1) include the well-known aromatic, araliphatic, aliphatic or cycloaliphatic polyisocyanates of an NCO functionality of >2.

Examples of such suitable polyisocyanates are 1,4-butylene diisocyanate, 1,6-hexamethylene diisocyanate (HDI), isophorone diisocyanate (IPDI), 2,2,4 and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 1,4-phenylene diisocyanate, 2,4- and/or 2,6-toluoylene diisocyanate, 1,5-naphthalene diisocyanate, 2,2′- and/or 2,4′- and/or 4,4′-diphenylmethane diisocyanate, 1,3- and/or 1,4-bis-(2-isocyanatoprop-2-yl)benzene (TMXDI), 1,3-bis-(isocyanatomethyl)benzene (XDI), and also alkyl 2,6-diisocyanatohexanoates (lysine diisocyanates) having C₁-C₈-alkyl groups.

As well as the aforementioned polyisocyanates, it is also possible to use, proportionally, modified diisocyanates of uretdione, isocyanurate, urethane, allophanate, biuret, iminooxadiazinedione and/or oxadiazinetrione structure and also non-modified polyisocyanate having more than 2 NCO groups per molecule, for example 4-isocyanatomethyl-1,8-octane diisocyanate (nonane triisocyanate) or triphenylmethane 4,4′,4″-triisocyanate.

Preferably, the polyisocyanates or polyisocyanate mixtures of the aforementioned kind have exclusively aliphatically and/or cycloaliphatically attached isocyanate groups and an average NCO functionality in the range from 2 to 4, preferably in the range from 2 to 2.6 and more preferably in the range from 2 to 2.4 for the mixture.

It is particularly preferable for A1) to utilize 1,6-hexamethylene diisocyanate, isophorone diisocyanate, the isomeric bis(4,4′-isocyanatocyclohexyl)methanes, and also mixtures thereof. A2) utilizes polymeric polyols having a number average molecular weight Mₙ in the range from 400 to 8000 g/mol, preferably from 400 to 6000 g/mol and more preferably from 600 to 3000 g/mol. These preferably have an OH functionality in the range from 1.5 to 6, more preferably in the range from 1.8 to 3 and most preferably in the range from 1.9 to 2.1.

Such polymeric polyols are the well-known polyurethane coating technology polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be used in A2) individually or in any desired mixtures with one another.

Such polyester polyols are the well-known polycondensates formed from di- and also optionally tri- and tetraols and di- and also optionally tri and tetracarboxylic acids or hydroxy carboxylic acids or lactones. Instead of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, also 1,2-propanediol, 1,3-propanediol, butanediol(1,3), butanediol(1,4), hexanediol(1,6) and isomers, neopentyl glycol or neopentyl glycol hydroxypivalic entyl glycol ester, of which hexanediol(1,6) and isomers, neopentyl glycol and entyl glycol ester, of which hexanediol(1,6) and isomers, neopentyl glycol and neopentyl glycol hydroxypivalate are preferred. Besides these it is also possible to use polyols such as trimethylolpropane, glycerol, erythritol, pentaerythritol, trimethylolbenzene or trishydroxyethyl isocyanurate.

Useful dicarboxylic acids include phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethyl glutaric acid and/or 2,2-dimethylsuccinic acid. The corresponding anhydrides can also be used as a source of an acid.

When the average functionality of the polyol to be esterified is >than 2, monocarboxylic acids, such as benzoic acid and hexanecarboxylic acid can be used as well in addition.

Preferred acids are aliphatic or aromatic acids of the aforementioned kind. Adipic acid, isophthalic acid and optionally trimellitic acid are particularly preferred.

Hydroxy carboxylic acids useful as reaction participants in the preparation of a polyester polyol having terminal hydroxyl groups include for example hydroxycaproic acid, hydroxybutyric acid, hydroxydecanoic acid, hydroxystearic acid and the like. Suitable lactones include caprolactone, butyrolactone and homologues. Caprolactone is preferred.

A2) may likewise utilize hydroxyl-containing polycarbonates, preferably polycarbonate diols, having number average molecular weights Mₙ in the range from 400 to 8000 g/mol and preferably in the range from 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, 1,2-propanediol, 1,3-propanediol, 1,3-butanediol, 1,4-butanediol, 1,6-hexanediol, 1,8-octanediol, neopentyl glycol, 1,4-bishydroxymethylcyclohexane, 2-methyl-1,3-propanediol, 2,2,4-trimethyl-1,3-pentane-diol, dipropylene glycol, polypropylene glycols, dibutylene glycol, polybutylene glycols, bisphenol A and lactone-modified diols of the aforementioned kind.

The polycarbonate diol preferably contains 40% to 100% by weight of hexanediol, preference being given to 1,6-hexanediol and/or hexanediol derivatives. Such hexanediol derivatives are based on hexanediol and have ester or ether groups as well as terminal OH groups. Such derivatives are obtainable by reaction of hexanediol with excess caprolactone or by etherification of hexanediol with itself to form di- or trihexylene glycol.

In lieu of or in addition to pure polycarbonate diols, polyether-polycarbonate diols can also be used in A2).

Hydroxyl-containing polycarbonates preferably have a linear construction.

A2) may likewise utilize polyether polyols.

Useful polyether polyols include for example the well-known polyurethane chemistry polytetramethylene glycol polyethers as are obtainable by polymerization of tetrahydro-furan by means of cationic ring opening.

Useful polyether polyols likewise include the well-known addition products of styrene oxide, ethylene oxide, propylene oxide, butylene oxides and/or epichlorohydrin onto di- or polyfunctional starter molecules. Polyether polyols based on the at least proportional addition of ethylene oxide onto di- or polyfunctional starter molecules can also be used as component A4) (nonionic hydrophilicizing agents).

Useful starter molecules include all prior art compounds, for example water, butyl diglycol, glycerol, diethylene glycol, trimethylolpropane, propylene glycol, sorbitol, ethylenediamine, triethanolamine, 1,4-butanediol. Preferred starter molecules are water, ethylene glycol, propylene glycol, 1,4-butanediol, diethylene glycol and butyl diglycol.

Particularly preferred embodiments of the polyurethane dispersions (I) contain as component A2) a mixture of polycarbonate polyols and polytetramethylene glycol polyols, the proportion of polycarbonate polyols in this mixture being in the range from 20% to 80% by weight and the proportion of polytetramethylene glycol polyols in this mixture being in the range from 80% to 20% by weight. Preference is given to a proportion of 30% to 75% by weight for polytetramethylene glycol polyols and to a proportion of 25% to 70% by weight for polycarbonate polyols. Particular preference is given to a proportion of 35% to 70% by weight for polytetramethylene glycol polyols and to a proportion of 30% to 65% by weight for polycarbonate polyols, each subject to the proviso that the sum total of the weight percentages for the polycarbonate and polytetramethylene glycol polyols is 100% and the proportion of component A2) which is accounted for by the sum total of the polycarbonate and polytetramethylene glycol polyether polyols is at least 50% by weight preferably 60% by weight and more preferably at least 70% by weight.

The compounds of component A3) have molecular weights from 62 to 400 g/mol.

A3) may utilize polyols of the specified molecular weight range with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol and also any desired mixtures thereof with one another.

Also suitable are ester diols of the specified molecular weight range such as α-hydroxy-butyl-ε-hydroxycaproic acid ester, ω-hydroxyhexyl-γ-hydroxybutyric acid ester, β-hydroxyethyl adipate or bis(β-hydroxyethyl)terephthalate.

A3) may further utilize monofunctional isocyanate-reactive hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferred compounds for component A3) are 1,6-hexanediol, 1,4-butanediol, neopentyl glycol and trimethylolpropane.

An anionically or potentially anionically hydrophilicizing compound for component A4) is any compound which has at least one isocyanate-reactive group such as a hydroxyl group and also at least one functionality such as for example —COO⁻M⁺, —SO₃⁻M⁺, —PO(O⁻M⁺)₂ where M⁺ is for example a metal cation, H, NH₄⁺, NHR₃⁺, where R in each occurrence may be C₁-C₁₂-alkyl, C₅-C₆-cycloalkyl and/or C₂-C₄-hydroxyalkyl, which functionality enters on interaction with aqueous media a pH-dependent dissociative equilibrium and thereby can have a negative or neutral charge. Useful anionically or potentially anionically hydrophilicizing compounds include mono- and dihydroxy carboxylic acids, mono- and dihydroxy sulphonic acids and also mono- and dihydroxy phosphonic acids and their salts. Examples of such anionic or potentially anionic hydrophilicizing agents are dimethylolpropionic acid, dimethylolbutyric acid, hydroxypivalic acid, malic acid, citric acid, glycolic acid, lactic acid and the propoxylated adduct formed from 2-butenediol and NaHSO₃ and described in DE-A 2 446 440, page 5-9, formula I-III. Preferred anionic or potentially anionic hydrophilicizing agents for component A4) are those of the aforementioned kind that have carboxylate or carboxyl groups and/or sulphonate groups.

Particularly preferred anionic or potentially anionic hydrophilicizing agents are those that contain carboxylate or carboxyl groups as ionic or potentially ionic groups, such as dimethylolpropionic acid, dimethylolbutyric acid and hydroxypivalic acid and salts thereof.

Useful nonionically hydrophilicizing compounds for component A4) include for example polyoxyalkylene ethers which contain at least one hydroxyl or amino group, preferably at least one hydroxyl group.

Examples are the monohydroxyl-functional polyalkylene oxide polyether alcohols containing on average 5 to 70 and preferably 7 to 55 ethylene oxide units per molecule and obtainable in a conventional manner by alkoxylation of suitable starter molecules (for example in Ullmanns Encyclopadie der technischen Chemie, 4th edition, volume 19, Verlag Chemie, Weinheim pages 31-38).

These are either pure polyethylene oxide ethers or mixed polyalkylene oxide ethers, containing at least 30 mol % and preferably at least 40 mol % of ethylene oxide units, based on all alkylene oxide units present.

Preferred polyethylene oxide ethers of the aforementioned kind are monofunctional mixed polyalkylene oxide polyethers having 40 to 100 mol % of ethylene oxide units and 0 to 60 mol % of propylene oxide units.

Preferred nonionically hydrophilicizing compounds for component A4) include those of the aforementioned kind that are block (co)polymers prepared by blockwise addition of alkylene oxides onto suitable starters.

Useful starter molecules for such nonionic hydrophilicizing agents include saturated monoalcohols such as methanol, ethanol, n-propanol, isopropanol, n-butanol, isobutanol, sec-butanol, the isomers pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anis alcohol or cinnamyl alcohol, secondary monoamines such as dimethylamine, diethylamine, dipropylamine, diisopropylamine, dibutylamine, bis(2-ethylhexyl)amine, N-methylcyclohexylamine, N-ethylcyclohexylamine or dicyclohexylamine and also heterocyclic secondary amines such as morpholine, pyrrolidine, piperidine or 1H pyrazole. Preferred starter molecules are saturated monoalcohols of the aforementioned kind. Particular preference is given to using diethylene glycol monobutyl ether or n-butanol as starter molecules.

Useful alkylene oxides for the alkoxylation reaction are in particular ethylene oxide and propylene oxide, which can be used in any desired order or else in admixture in the alkoxylation reaction.

Component B1) may utilize di- or polyamines such as 1,2-ethylenediamine, 1,2-diaminopropane, 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylenetriamine, triaminononane, 1,3-xylylenediamine, 1,4-xylylenediamine, α,α,α′,α′-tetramethyl-1,3- and -1,4-xylylenediamine and 4,4-diaminodicyclohexylmethane and/or dimethylethylenediamine. It is also possible but less preferable to use hydrazine and also hydrazides such as adipohydrazide.

Component B1) can further utilize compounds which as well as a primary amino group also have secondary amino groups or which as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Component B1) can further utilize monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide-amines formed from diprimary amines and monocarboxylic acids, monoketimes of diprimary amines, primary/tertiary amines, such as N,N-dimethylaminopropylamine.

Preferred compounds for component B1) are 1,2-ethylenediamine, 1,4-diaminobutane and isophoronediamine. Mixtures of the abovementioned diamines of component B1) are more preferably used, in particular mixtures of 1,2-ethylenediamine and isophoronediamine and also mixtures of 1,4-diaminobutane and isophoronediamine.

An anionically or potentially anionically hydrophilicizing compound for component B2) is any compound which has at least one isocyanate-reactive group, preferably an amino group, and also at least one functionality such as for example —COO⁻M⁺, —SO₃⁻M⁺, —PO(O⁻M⁺)₂ where M⁺ is for example a metal cation, H⁺, NH₄⁻, NHR₃⁺, where R in each occurrence may be C₁-C₁₂-alkyl, C₅-C₆-cycloalkyl and/or C₂-C₄-hydroxyalkyl, which functionality enters on interaction with aqueous media a pH-dependent dissociative equilibrium and thereby can have a negative or neutral charge.

Useful anionically or potentially anionically hydrophilicizing compounds are mono- and diamino carboxylic acids, mono- and diamino sulphonic acids and also mono- and diamino phosphonic acids and their salts. Examples of such anionic or potentially anionic hydrophilicizing agents are N-(2-aminoethyl)-β-alanine, 2-(2-aminoethylamino)ethanesulphonic acid, ethylenediaminepropylsulphonic acid, ethylenediaminebutylsulphonic acid, 1,2- or 1,3-propylenediamine-β-ethylsulphonic acid, glycine, alanine, taurine, lysine, 3,5-diaminobenzoic acid and the addition product of IPDA and acrylic acid (EP-A 0 916 647, Example 1). It is further possible to use cyclohexyl-aminopropanesulphonic acid (CAPS) from WO-A 01/88006 as anionic or potentially anionic hydrophilicizing agent.

Preferred anionic or potentially anionic hydrophilicizing agents for component B2) are those of the aforementioned kind that have carboxylate or carboxyl groups and/or sulphonate groups, such as the salts of N-(2-aminoethyl)-β-alanine, of 2-(2-aminoethylamino)ethanesulphonic acid or of the addition product of IPDA and acrylic acid (EP-A 0 916 647, Example 1).

Mixtures of anionic or potentially anionic hydrophilicizing agents and nonionic hydrophilicizing agents can also be used.

A preferred embodiment for producing the specific polyurethane dispersions utilizes components A1) to A4) and B1) to B2) in the following amounts, the individual amounts always adding up to 100% by weight:

5% to 40% by weight of component A1),
55% to 90% by weight of A2),
0.5% to 20% by weight of the sum total of components A3) and B1)
0.1% to 25% by weight of the sum total of the components component A4) and B2), with 0.1% to 5% by weight of anionic or potentially anionic hydrophilicizing agents from A4) and/or B2) being used, based on the total amount of components A1) to A4) and B1) to B2).

A particularly preferred embodiment for producing the specific polyurethane dispersions utilizes components A1) to A4) and B1) to B2) in the following amounts, the individual amounts always adding up to 100% by weight:

5% to 35% by weight of component A1),
60% to 90% by weight of A2),
0.5% to 15% by weight of the sum total of components A3) and B1)
0.1% to 15% by weight of the sum total of the components component A4) and B2), with 0.2% to 4% by weight of anionic or potentially anionic hydrophilicizing agents from A4) and/or B2) being used, based on the total amount of components A1) to A4) and B1) to B2).

A very particularly preferred embodiment for producing the specific polyurethane dispersions utilizes components A1) to A4) and B1) to B2) in the following amounts, the individual amounts always adding up to 100% by weight:

10% to 30% by weight of component A1),
65% to 85% by weight of A2),
0.5% to 14% by weight of the sum total of components A3) and B1)
0.1% to 13.5% by weight of the sum total of the components A4) and B2), with 0.5% to 3.0% by weight of anionic or potentially anionic hydrophilicizing agents from A4) and/or B2) being used, based on the total amount of components A1) to A4) and B1) to B2).

The production of the anionically hydrophilicized polyurethane dispersions (I) can be carried out in one or more stages in homogeneous phase or, in the case of a multistage reaction, partly in disperse phase. After completely or partially conducted polyaddition from A1) to A4) a dispersing, emulsifying or dissolving step is carried out. This is followed if appropriate by a further polyaddition or modification in disperse phase.

Any prior art process can be used, examples being the prepolymer mixing process, the acetone process or the melt dispersing process. The acetone process is preferred.

Production by the acetone process typically involves the constituents A2) to A4) and the polyisocyanate component A1) being wholly or partly introduced as an initial charge to produce an isocyanate-functional polyurethane prepolymer and optionally diluted with a water-miscible but isocyanate-inert solvent and heated to temperatures in the range from 50 to 120° C. The isocyanate addition reaction can be speeded using the catalysts known in polyurethane chemistry.

Useful solvents include the customary aliphatic, keto-functional solvents such as acetone, 2-butanone, which can be added not just at the start of the production process but also later, optionally in portions. Acetone and 2-butanone are preferred.

Other solvents such as xylene, toluene, cyclohexane, butyl acetate, methoxypropyl acetate, N-methylpyrrolidone, N-ethylpyrrolidone, solvents having ether or ester units can additionally be used or wholly or partly distilled off or in the case of N-methylpyrrolidone, N-ethylpyrrolidone remain completely in the dispersion. But preference is given to not using any other solvents apart from the customary aliphatic, keto-functional solvents.

Subsequently, any constituents of A1) to A4) not added at the start of the reaction are added.

In the production of the polyurethane prepolymer from A1) to A4), the amount of substance ratio of isocyanate groups to with isocyanate-reactive groups is in the range from 1.05 to 3.5, preferably in the range from 1.2 to 3.0 and more preferably in the range from 1.3 to 2.5.

The reaction of components A1) to A4) to form the prepolymer is effected partially or completely, but preferably completely. Polyurethane prepolymers containing free isocyanate groups are obtained in this way, without a solvent or in solution.

The neutralizing step to effect partial or complete conversion of potentially anionic groups into anionic groups utilizes bases such as tertiary amines, for example trialkylamines having 1 to 12 and preferably 1 to 6 carbon atoms and more preferably 2 to 3 carbon atoms in every alkyl radical or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may also bear for example hydroxyl groups, as in the case of the dialkylmonoalkanol-, alkyldialkanol- and trialkanolamines. Useful neutralizing agents further include if appropriate inorganic bases, such as aqueous ammonia solution, sodium hydroxide or potassium hydroxide.

Preference is given to ammonia, triethylamine, triethanolamine, dimethylethanolamine or diisopropylethylamine and also sodium hydroxide and potassium hydroxide, particular preference being given to sodium hydroxide and potassium hydroxide.

The bases are employed in an amount of substance which is between 50 and 125 mol % and preferably between 70 and 100 mol % of the amount of substance of the acid groups to be neutralized. Neutralization can also be effected at the same time as the dispersing step, by including the neutralizing agent in the water of dispersion.

Subsequently, in a further process step, if this has not already been done or only to some extent, the prepolymer obtained is dissolved with the aid of aliphatic ketones such as acetone or 2-butanone.

In the chain extension of stage B), NH₂— and/or NH-functional components are reacted, partially or completely, with the still remaining isocyanate groups of the prepolymer. Preferably, the chain extension/termination is carried out before dispersion in water.

Chain termination is typically carried out using amines B1) having an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl-(methyl)aminopropylamine, morpholine, piperidine or suitable substituted derivatives thereof, amide-amines formed from diprimary amines and monocarboxylic acids, monoketimes of diprimary amines, primary/tertiary amines, such as N,N-dimethylaminopropylamine.

When partial or complete chain extension is carried out using anionic or potentially anionic hydrophilicizing agents conforming to definition B2) with NH₂ or NH groups, chain extension of the prepolymers is preferably carried out before dispersion.

The aminic components B1) and B2) can optionally be used in water- or solvent-diluted form in the process of the present invention, individually or in mixtures, any order of addition being possible in principle.

When water or organic solvent is used as a diluent, the diluent content of the chain-extending component used in B) is preferably in the range from 70% to 95% by weight.

Dispersion is preferably carried out following chain extension. For dispersion, the dissolved and chain-extended polyurethane polymer is either introduced into the dispersing water, if appropriate by substantial shearing, such as vigorous stirring for example, or conversely the dispersing water is stirred into the chain-extended polyurethane polymer solutions. It is preferable to add the water to the dissolved chain-extended polyurethane polymer.

The solvent still present in the dispersions after the dispersing step is then typically removed by distillation. Removal during the dispersing step is likewise possible.

The residual level of organic solvents in the polyurethane dispersions (I) is typically less than 1.0% by weight and preferably less than 0.5% by weight, based on the entire dispersion.

The pH of the polyurethane dispersions (I) which are essential to the present invention is typically less than 9.0, preferably less than 8.5, more preferably less than 8.0 and most preferably is in the range from 6.0 to 7.5.

The solids content of the polyurethane dispersions (I) is preferably in the range from 40% to 70%, more preferably in the range from 50% to 65%, most preferably in the range from 55% to 65% and in particular in the range from 60% to 65% by weight.

Any water-soluble inorganic salts known per se to one skilled in the art can be used as inorganic, cationic coagulants (II) in the compositions. Preferred inorganic, cationic coagulants (II) are alkali metal and alkaline earth metal salts and also salts of group 13 of the periodic table. Particular preference is given to the salts of alkaline earth metals and very particular preference to the salts of magnesium and calcium. Preference is given to using the chlorides, sulphates and phosphates of the above-described metal ions. Magnesium chloride, calcium chloride and sodium chloride may be mentioned specifically.

The inorganic, cationic coagulants can be used in solid form or as aqueous solutions. The use of aqueous solutions is preferred.

As well as the polyurethane dispersions (I) and the inorganic coagulants (II), auxiliary and additive materials (III) can also be used.

Examples of such auxiliary and additive materials (III) are foam auxiliaries such as foam formers and stabilizers, thickeners or thixotroping agents, antioxidants, light stabilizers, emulsifiers, plasticizers, pigments, fillers and/or flow control agents.

Preferably, foam auxiliaries such as foam formers and stabilizers are included as auxiliary and additive materials (III). Useful foam auxiliaries include commercially available compounds such as fatty acid amides, sulphosuccinamides hydrocarbyl sulphates or sulphonates or fatty acid salts, in which case the lipophilic radical preferably contains 12 to 24 carbon atoms, and also alkyl polyglycosides obtainable in a conventional manner by reaction of comparatively long-chain monoalcohols (4 to 22 carbon atoms in the alkyl radical) with mono-, di- or polysaccharides (see for example Kirk-Othmer, Encyclopedia of Chemical Technology, John Wiley & Sons, Vol. 24, p. 29). Particularly suitable foam auxiliaries are EO-PO block copolymers obtainable in a conventional manner by addition of ethylene oxide and propylene oxide onto OH— or NH— functional starter molecules (see for example Kirk-Othmer, Encyclopedia of Chemical Technology, John Wiley & Sons, Vol. 24, p. 28). To improve foam formation, foam stability or the properties of the resulting polyurethane foam further additives may be present in component (III) as well as the EO-PO block copolymers. Such further additives may in principle be any anionic, nonionic or cationic surfactant known per se. Preferably, however, only the EO-PO block copolymers are used as component (III).

Commercially available thickeners can be used, such as derivatives of dextrin, of starch, of polysaccharide such as gum arabic or cellulose derivatives, examples being cellulose ethers or hydroxyethylcellulose, organic wholly synthetic thickeners based on polyacrylic acids, polyvinylpyrrolidones, polymethacrylic compounds or polyurethanes (associative thickeners) and also inorganic thickeners, such as bentonites or silicas.

In principle, although not preferably, the compositions which are essential to the present invention can also contain crosslinkers such as unblocked polyisocyanates, amide- and amine-formaldehyde resins, phenolic resins, aldehydic and ketonic resins, examples being phenol-formaldehyde resins, resols, furan resins, urea resins, carbamic ester resins, triazine resins, melamine resins, benzoguanamine resins, cyanamide resins or aniline resins.

The compositions which are essential to the present invention typically contain, based on dry substance, 85 to 99.5 parts by weight of dispersion (I), 0.5 to 15 parts by weight of inorganic cationic coagulant (II), 0 to 10 parts by weight of foam auxiliary, 0 to 10 parts by weight of crosslinker and 0 to 15 parts by weight of thickener.

Preferably, the compositions which are essential to the present invention contain, based on the dry substance, 85 to 99.5 parts by weight of dispersion (I), 0.5 to 15 parts by weight of inorganic cationic coagulant (II), 1 to 7.5 parts by weight of foam auxiliary, 0 to 5 parts by weight of crosslinker and 1 to 15 parts by weight of thickener, more preferably, 70 to 99.5 parts by weight of dispersion (I), 0.5 to 15 parts by weight of inorganic cationic coagulant (II), 2.5 to 7.5 parts by weight of foam auxiliary and 1 to 15 parts by weight of thickener (based on the dry substance).

As well as components (I), (II) and if appropriate (III), other aqueous binders can also be used in the compositions which are essential to the present invention. Such aqueous binders can be constructed for example of polyester, polyacrylate, polyepoxy or other polyurethane polymers. Similarly, the combination with radiation-curable binders as described for example in EP-A-0 753 531 is also possible. It is further possible to employ other anionic or nonionic dispersions, such as polyvinyl acetate, polyethylene, polystyrene, polybutadiene, polyvinyl chloride, polyacrylate and copolymer dispersions.

Frothing in the process of the present invention is accomplished by mechanical stirring of the composition at high speeds of rotation by shaking or by decompressing a blowing gas.

Mechanical frothing can be effected using any desired mechanical stirring, mixing and dispersing techniques. Air is generally introduced, but nitrogen and other gases can also be used for this purpose.

The foam thus obtained is, in the course of frothing or immediately thereafter, applied to a substrate or introduced into a mould and dried.

Application to a substrate can be for example by pouring or blade coating, but other conventional techniques are also possible. Multilayered application with intervening drying steps is also possible in principle.

A satisfactory drying rate for the foams is observed at a temperature as low as 20° C., so that drying on injured human or animal tissue presents no problem. However, temperatures above 30° C. are preferably used for more rapid drying and fixing of the foams. However, drying temperatures should not exceed 200° C., preferably 150° C. and more preferably 130° C., since undesirable yellowing of the foams can otherwise occur, inter alia. Drying in two or more stages is also possible.

Drying is generally effected using conventional heating and drying apparatus, such as (circulating air) drying cabinets, hot air or IR radiators. Drying by leading the coated substrate over heated surfaces, for example rolls, is also possible.

Application and drying can each be carried out batchwise or continuously, but the entirely continuous process is preferred.

Useful substrates include in particular papers or films which facilitate simple detachment of the wound dressing material before it is used to cover an injured site. Human or animal tissue such as skin can similarly serve as a substrate, so that direct closure of an injured site is possible by a wound dressing produced in situ.

The present invention further provides the wound dressings obtainable by the process of the present invention.

Before drying, the foam densities of the polyurethane foams are typically in the range from 50 to 800 g/litre, preferably in the range from 100 to 500 g/litre.

After drying, the polyurethane foams have a microporous, at least partial open-cell structure comprising intercommunicating cells. The density of the dried foams is typically below 0.4 g/cm³, preferably below 0.35 g/cm³ and more preferably in the range from 0.01 to 0.3 g/cm³.

The DIN EN 13726-1 Part 3.2 physiological saline absorbency is typically in the range from 100 to 1500% and preferably in the range from 300 to 1500% for the polyurethane foams (mass of liquid taken up, based on the mass of dry foam). The DIN EN 13726-2 Part 3.2 water vapour transmission rate is typically in the range from 500 to 8000 g/24 h*m² and is preferably in the range from 500 to 5000 g/24 h*m².

The polyurethane foams exhibit good mechanical strength and high elasticity. Typically, maximum stress is greater than 0.2 N/mm² and elongation at break is greater than 200%, preferably, elongation at break is greater than 250% (determined according to DIN 53504).

After drying, the thickness of the polyurethane foams is typically in the range from 0.1 mm to 50 mm, preferably in the range from 0.5 mm to 20 mm.

The polyurethane foams can moreover be adhered, laminated or coated to or with further materials, for example materials based on hydrogels, (semi-) permeable films, coatings, hydrocolloids or other foams.

If appropriate, a sterilizing step can be included in the process of the present invention. It is similarly possible in principle for the wound dressings obtainable by the process of the present invention to be sterilized after they have been produced. Conventional sterilizing processes are used where sterilization is effected by thermal treatment suitable chemicals such as ethylene oxide or irradiation with gamma rays for example.

It is likewise possible to add, incorporate or coat with antimicrobially or biologically active components which for example have a positive effect with regard to wound healing and the avoidance of germ loads.

Preferred active components of the aforementioned kind are those from the group consisting of antiseptics, growth factors, protease inhibitors and nonsteroidal anti-inflammatories/opiates.

In a preferred embodiment of the present invention, the active component comprises an antiseptic biguanide and/or its salt, preferably the hydrochloride.

Biguanides are compounds derived from biguanide (C₂H₇N₅), in particular its polymers. Antiseptic biguanides are biguanides that have an antimicrobial effect, i.e. act as bacteriostats or preferably as bactericides. The compounds in question preferably have a broad effect against many bacteria and can be characterized by a minimal microbicidal concentration (MMC, measured in the suspension test) of at least 0.5 μg/ml, preferably at least 12 or at least 25 μg/ml with regard to E. coli.

A preferred antiseptic biguanide according to this invention is poly(imino[iminocarbonyl]iminopolymethylene), the use of poly(hexamethylene)-biguanide (PHMB), also known as polyhexanide, as antiseptic biguanide being particularly preferred.

The term “antiseptic biguanides” according to this invention also comprehends metabolites and/or prodrugs of antiseptic biguanides. Antiseptic biguanides can be present as racemates or pure isoforms.

The foamed articles of polyurethane foams or the compositions according to the present invention preferably contain antiseptic biguanide and/or its salt, preferably the hydrochloride, in a concentration of 0.01% to 20% by weight, the concentration of 0.1% to 5% by weight being particularly advantageous. The biguanide may have any desired molecular weight distribution.

Owing to the wide utility of the process of the present invention and of the foams and wound dressings obtainable thereby, it is possible in principle to use said process in the industrial production of foams and wound dressings. But it is similarly also possible to use it for producing sprayed plasters for example, in which case the wound dressing is formed by direct application of the composition to a wound and simultaneous frothing and subsequent drying.

For industrial production of foams and wound dressings, the polyurethane dispersion (I) is mixed with foam auxiliaries of the aforementioned kind and thereafter mechanically frothed by introduction of a gas such as air and finally coagulated by addition of the inorganic, cationic coagulant (II), to obtain a further processible, coagulated foam. This foam is applied to a substrate and dried. Owing to higher productivity, drying is typically carried out at elevated temperatures in the range from 30 to 200° C., preferably in the range from 50 to 150° C. and more preferably in the range from 60 to 130° C. Drying is generally carried out using conventional heating and drying apparatus, for example (circulating air) drying cabinets. Application and drying can each be carried out batchwise or continuously, but preference is given to the wholly continuous process.

When the composition which is essential to the present invention is used to produce a sprayed plaster, the polyurethane dispersion (I) and the inorganic, cationic coagulant (II), which each may contain foam auxiliaries if appropriate, are separately provided and then mixed with each other immediately before or during application to the tissue to be covered. Frothing is here accomplished by simultaneous decompression of a blowing gas which was present in at least one of the components (I) and (II). To consolidate the foam formed, it is subsequently dried, for which temperatures of 20 to 40° C. are sufficient. When additional heat sources such as a hair dryer or an IR red light lamp are used, forced thermal drying up to a maximum temperature of 80° C. is also possible.

The blowing agents used are well known from polyurethane chemistry. n-Butane, i-butane and propane and also mixtures thereof are suitable for example, as is also dimethyl ether for example. Preference is given to using a mixture of n-butane, i-butane and propane, whereby the desired, fine-cell foams are obtained. The blowing agent or blowing agent mixture is typically used in an amount of 1% to 50% by weight, preferably 5% to 40% by weight and more preferably 5% to 20% by weight, the sum total of polyurethane dispersion (I) used, inorganic, cationic coagulant (II), blowing agent (mixture) and also optional auxiliary and adjunct materials (III) being 100% by weight. Spray plasters are preferably provided in spray cans, the polyurethane dispersion (I) and the inorganic, cationic coagulant (II) being included separately from each other and not being mixed with each other until immediately before application. The blowing agent can be included in either or both of the components. Either or both of the components (I) and (II) may additionally if appropriate also contain auxiliary and additive materials (III), preferably foam auxiliaries. Pouring of the composition is possible as well as spraying.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

Unless indicated otherwise, all percentages are by weight.

Solids contents were determined in accordance with DIN-EN ISO 3251.

NCO contents were unless expressly mentioned otherwise determined volumetrically in accordance with DIN-EN ISO 111909.

### Substances and Abbreviations Used:

Diaminosulphonate: NH₂—CH₂CH₂—NH—CH₂CH₂—SO₃Na (45% in water)
Desmophen® C2200: polycarbonate polyol, OH number 56 mg KOH/g, number average molecular weight 2000 g/mol (Bayer MaterialScience AG, Leverkusen, Germany)
PolyTHF® 2000: polytetramethylene glycol polyol, OH number 56 mg KOH/g, number average molecular weight 2000 g/mol (BASF AG, Ludwigshafen, Germany)
PolyTHF® 1000: polytetramethylene glycol polyol, OH number 112 mg KOH/g, number average number average molecular weight 1000 g/mol (BASF AG, Ludwigshafen, Germany)
LB 25 polyether: monofunctional polyether based on ethylene oxide/propylene oxide, number average molecular weight 2250 g/mol, OH number 25 mg KOH/g (Bayer MaterialScience AG, Leverkusen, Germany)
Impranil® DLN: aliphatic polyester polyurethane dispersion, solids content 40%, pH 6.6 (BayerMaterialScience AG, Leverkusen, Germany)

The determination of the average particle sizes (the number average is reported) of the polyurethane dispersions (I) was carried out using laser correlation spectroscopy (instrument: Malvern Zetasizer 1000, Malver Inst. Limited).

### Example 1

### Polyurethane Dispersion 1

987.0 g of PolyTHF® 2000, 375.4 g of PolyTHF® 1000, 761.3 g of Desmophen® C2200 and 44.3 g of LB 25 polyether were heated to 70° C. in a standard stirring apparatus. Then, a mixture of 237.0 g of hexamethylene diisocyanate and 313.2 g of isophorone diisocyanate was added at 70° C. in the course of 5 min and the mixture was stirred at 120° C. until the theoretical NCO value was reached or the actual NCO value was slightly below the theoretical NCO value. The ready-produced prepolymer was dissolved with 4830 g of acetone and, in the process, cooled down to 50° C. and subsequently admixed with a solution of 25.1 g of ethylenediamine, 116.5 g of isophoronediamine, 61.7 g of diaminosulphonate and 1030 g of water metered in over 10 min. The mixture was subsequently stirred for 10 min. Then, a dispersion was formed by addition of 1250 g of water. This was followed by removal of the solvent by distillation under reduced pressure.

The white dispersion obtained had the following properties:

| | |
|---|---|
| Solids content: | 61% |
| Particle size (LKS): | 312 nm |
| Viscosity (viscometer, 23° C.): | 241 mPas |
| pH (23° C.): | 6.02 |

### Example 2

### Polyurethane Dispersion 2

1077.2 g of PolyTHF® 2000, 409.7 g of PolyTHF® 1000, 830.9 g of Desmophen® C2200 and 48.3 g of LB 25 polyether were heated to 70° C. in a standard stirring apparatus. Then, a mixture of 258.7 g of hexamethylene diisocyanate and 341.9 g of isophorone diisocyanate was added at 70° C. in the course of 5 min and the mixture was stirred at 120° C. until the theoretical NCO value was reached or the actual NCO value was slightly below the theoretical NCO value. The ready-produced prepolymer was dissolved with 4840 g of acetone and, in the process, cooled down to 50° C. and subsequently admixed with a solution of 27.4 g of ethylenediamine, 127.1 g of isophoronediamine, 67.3 g of diaminosulphonate and 1200 g of water metered in over 10 min. The mixture was subsequently stirred for 10 min. Then, a dispersion was formed by addition of 654 g of water. This was followed by removal of the solvent by distillation under reduced pressure.

The polyurethane dispersion obtained had the following properties:

| | |
|---|---|
| Solids content: | 61.6% |
| Particle size (LKS): | 528 nm |
| Viscosity (viscometer, 23° C.): | 166 mPas |
| pH (23° C.): | 7.5 |

### Examples 3-12

Foams produced from the polyurethane dispersions of Examples 1-2 and also Impranil® DLN

The Table 1 amounts of the commercially available polyurethane dispersion Impranil® DLN or of the polyurethane dispersions produced as described in Examples 1 and 2 were mixed with the foam auxiliaries and also the thickener indicated in Table 1 and frothed by means of a commercially available hand stirrer (stirrer made of bent wire) for 4 minutes. While stirring was continued, the foams obtained were finally coagulated by addition of the salt solution indicated in Table 1; coagulation left foam volume practically unchanged. Within a few seconds solid foams resulted with good mechanical properties. The subsequent drying of the solidified foams was carried out overnight at room temperature. Clean white hydrophilic foams having a fine porous structure were obtained without exception.

TABLE 1

| | Amount [g] | | | | |
|---|---|---|---|---|---|
| | Polyurethane | Foam | Foam | Coagulant | |
| Foam | dispersion | additive | additive | (concentration | Gum |
| No. | (Example) | 1 | 2 | in water) | arabic |
| 1 | 40.0 (1) | 0.5¹⁾ | — | 1.1 (70%)⁴⁾ | 0.8 |
| 2 | 40.0 (2) | 0.6¹⁾ | — | 1.2 (70%)⁴⁾ | 0.8 |
| 3 | 40.0 (1) | 1.0²⁾ | 0.1³⁾ | 1.2 (70%)⁴⁾ | 0.9 |
| 4 | 40.0 (2) | 1.1²⁾ | 0.1³⁾ | 1.3 (70%)⁵⁾ | 0.9 |
| 5 | 40.0 (1) | 0.5¹⁾ | — | 1.2 (70%)⁶⁾ | 0.8 |
| 6 | 40.0 (1) | 0.5¹⁾ | — | 1.2 (70%)⁶⁾ | 0.4 |
| 7 | 40.0 | 0.5¹⁾ | — | 2.3 (33%)⁵⁾ | 0.8 |
| | (Impranil ® DLN) | | | | |
| 8 | 40.0 | 0.5¹⁾ | — | 2.9 (26%)⁷⁾ | 0.8 |
| | (Impranil ® DLN) | | | | |
| 9 | 40.0 | 0.5¹⁾ | — | 2.6 (29%)⁸⁾ | 0.7 |
| | (Impranil ® DLN) | | | | |
| 10 | 40.0 | 0.5¹⁾ | — | 2.3 (33%)⁵⁾ | — |
| | (Impranil ® DLN) | | | | |

¹⁾EO-PO block copolymer (Pluronic ® PE 6800, BASF AG, Ludwigshafen, Germany);
²⁾alkyl polyglycoside (Plantacare ® 1200 UP, Cognis Deutschland GmbH & Co. KG, Düsseldorf, Germany);
³⁾ammonium stearate (about 30%, Stokal ® STA, Bozzetto GmbH, Krefeld, Germany);
⁴⁾MgCl₂;
⁵⁾MgSO₄;
⁶⁾CaCl₂;
⁷⁾NaCl;
⁸⁾Al₂(SO₄)₃

### Comparative Example 1

### Foam Production Test without Coagulation

40.0 g of Impranil® DLN were mixed with 0.5 g of Pluronic® PE 6800 and 0.8 g of gum arabic and whipped for 4 minutes using a commercially available manual stirring device (a stirrer made of bent wire). Then the foam was spread onto silicone-coated paper. During the subsequent drying of the foam at room temperature the foam collapsed and a coarsely porous foam sheet was obtained.

### Comparative Example 2

### Foam Production Test without Coagulation

40.0 g of Impranil® DLN were mixed with 0.5 g of Pluronic® PE 6800 and whipped for 4 minutes using a commercially available manual stirring device (a stirrer made of bent wire). Then the foam was spread onto silicone-coated paper. During the subsequent drying of the foam at room temperature the foam collapsed and a coarsely porous foam sheet was obtained.

1. A process for producing foamed articles made of polyurethane foams comprising frothing and drying a composition comprising an aqueous, anionically hydrophilicized, polyurethane dispersion (I) and an inorganic cationic coagulant (II).

2. The process of claim 1, wherein said foamed article is a wound contact material.

3. The process of claim 1, wherein said aqueous, anionically hydrophilicized, polyurethane dispersion (I) is prepared by
A) producing isocyanate-functional prepolymers from
A1) organic polyisocyanates;
A2) polymeric polyols having number-average molecular weights in the range from 400 to 8000 g/mol and OH functionalities in the range from 1.5 to 6; and
A3) optionally hydroxyl-functional compounds having molecular weights in the range from 62 to 399 g/mol; and
A4) optionally isocyanate-reactive, anionic or potentially anionic and optionally nonionic hydrophilicizing agents and
B) wholly or partly reacting the free NCO groups of said isocyanate-functional prepolymer
B1) optionally with amino-functional compounds having molecular weights in the range from 32 to 400 g/mol; and
B2) with amino-functional, anionic or potentially anionic hydrophilicizing agents;
by chain extension;
wherein said isocyanate-functional prepolymers are dispersed in water before, during, or after step B), and
wherein any potentially ionic groups present are converted into the ionic form by partial or complete reaction with a neutralizing agent.

4. The process of claim 3, wherein A1) is 1,6-hexamethylene diisocyanate, isophorone diisocyanate, the isomeric bis-(4,4′-isocyanatocyclohexyl)methanes, or mixtures thereof, and A2) is a mixture of polycarbonate polyols and polytetramethylene glycol polyols, wherein the proportion of A2) which is contributed by the sum total of the polycarbonate and polytetramethylene glycol polyether polyols is at least 70% by weight.

5. The process of claim 1, wherein said inorganic cationic coagulant (II) is a water-soluble inorganic salt.

6. The process of claim 5, wherein said water-soluble inorganic salt is an alkaline earth metal salt.

7. The process of claim 6, wherein said water-soluble inorganic salt is magnesium chloride, calcium chloride, or a mixture thereof.

8. The process of claim 1, further comprising auxiliary and additive materials (III).

9. The process of claim 8, wherein said auxiliary and additive materials (III) foam formers and stabilizers selected from the group consisting of fatty acid amides, sulphosuccinamides, hydrocarbyl sulphonates, hydrocarbyl sulphates, fatty acid salts, EO-PO block copolymers and/or alkyl polyglycoside.

10. The process of claim 8, wherein said foam formers and stabilizers are EO-PO block copolymers.

11. The process of claim 1, wherein said, further comprising active components selected from the group consisting of antiseptics, growth factors, protease inhibitors, and nonsteroidal anti-inflammatories/opiates.

12. The process of claim 11, wherein said active component is an antiseptic biguanide and/or its salt.

13. A foamed article produced by the process of claim 1.

14. The foamed article of claim 13, wherein said foamed article has a microporous, open-cell structure and a density of below 0.4 g/cm³ in the dried state.

15. The foamed article of claim 13, wherein said foamed article has a DIN EN 13726-1 Part 3.2 physiological saline absorbency of 100 to 1500% (mass of liquid taken up, based on the mass of dry foam) and a DIN EN 13726-2 Part 3.2 water vapour transmission rate in the range from 500 to 8000 g/24 h*m².

16. The foamed article of claim 13, wherein said foamed article is a wound contact material.

17. A composition comprising an aqueous, anionically hydrophilicized polyurethane dispersion (I) and an inorganic cationic coagulant (II).

18. The composition of claim 17, further comprising an active component selected from the group consisting of antiseptics, growth factors, protease inhibitors, and nonsteroidal anti-inflammatories/opiates.

19. The composition of claim 18, wherein said active component is an antiseptic biguanide and/or its salt.
